(19) United States
(12) Patent Application Publication
(10) Pub. No.: US 2023/0059045 A1
(43) Pub. Date: Feb. 23, 2023

(54) COUPLING DEVICE FOR MEDICAL TUBING

(71) Applicant: CAREFUSION 303, INC., San Diego, CA (US)

(72) Inventors: Raymond P. FEITH, Chino Hills, CA (US); Jason Andrew WINE, Brea, CA (US)

(21) Appl. No.: 17/885,477

(22) Filed: Aug. 10, 2022

Related U.S. Application Data
(60) Provisional application No. 63/235,120, filed on Aug. 19, 2021.

(51) Int. Cl. A61M 39/10

(52) U.S. Cl. CPC A61M 39/1011; A61M 39/20; A61M 2039/1033

(57) ABSTRACT

A coupling device that can removably couple medical tubing with a mating connector is disclosed and can include a housing and an inner body movable to different orientations, where an orientation is configured to resist separation of the coupling device and mating connector, and another orientation is configured such that resistance to separation from the mating connector is reduced, where any of the housing and the inner body can be moved relative to each other, and where the coupling device can permit separation of the coupling device and mating connector when a portion of the coupling device is moved, biased, and/or flexed when the coupling device and the mating connector are moved in directions away from each other, and where the coupling device can be re-coupled with a mating connector after separation of the coupling device and the mating connector.

### CROSS-REFERENCE TO RELATED APPLICATIONS

This application claims the benefit of U.S. Provisional Application No. 63/235,120, titled “COUPLING DEVICE FOR MEDICAL TUBING,” filed Aug. 19, 2021, the disclosure of which is incorporated herein by reference in its entirety.

### BACKGROUND

The present disclosure relates generally to medical fluid connectors and, more particularly, to coupling devices for connecting and disconnecting medical tubing.

Medical connections are widely used in fluid delivery systems such as those used in connection with intravenous (IV) fluid lines, blood access, hemodialysis, peritoneal dialysis, enteral feeding, drug vial access, and other procedures.

In some instances, the medical connection can become dislodged or disconnected in an unintended manner. For example, medical tubing of an IV set that is coupled to a catheter can become dislodged when an unintended or unexpected forces is exerted upon the catheter, which may exceed the design limitations of the catheter securement method. An unintended or unexpected force can be applied to the tubing and/or catheter when the patient moves or rolls over within a bed, or when the tubing or another portion of an intravenous set become caught on a portion of the bed, such as the railing, or when a patient is panicking, disoriented, or fidgeting to such an extent that the medical tubing is unintentionally or intentionally pulled away from the patient or away from the medical equipment coupled to the tubing.

### SUMMARY

In accordance with at least some embodiments disclosed herein is the realization that unintended dislodgement or disconnection of a medical connection, such as a medical fluid line, can result in injury to a patient or a caregiver, such as by depriving the patient of a medicament, increasing the potential for infection to the patient, and exposing the caregiver to harmful medicaments.

Accordingly, an aspect of the present disclosure provides a coupling device for medical tubing, the coupling device comprising: a housing having an inner surface forming a cavity, a first opening into the cavity, and a second opening into the cavity; and an inner body having a fluid passage, extending through the inner body, and a coupling sleeve, wherein the inner body is positioned within the cavity of the housing with the coupling sleeve extending toward the first opening of the housing such that the housing and the inner body are movable relative to each other; wherein, in a first position of the inner body, biasing of at least a portion of the coupling sleeve in a direction that is radially outward is resisted by engagement of the coupling sleeve with the housing; and wherein, in a second position of the inner body, the coupling sleeve is moved through the first opening in a direction that is away from the second opening of the housing such that the at least a portion of the coupling sleeve can be biased radially outward.

Some instances of the present disclosure provide a fluid-fitting system comprising a coupling device for medical tubing, the coupling device comprising: a housing having a first end, a second end, and an inner surface forming a cavity, wherein the first end comprises a first opening into the cavity, and the second end comprises a second opening into the cavity; an inner body having a first end portion, a second end portion, a fluid passage extending through the first and second end portions of the inner body, the first end portion comprising a coupling sleeve formed by a wall having an inner surface that extends around a longitudinal axis defined by the fluid passage to form a recess extending from a first end of the inner body toward the second end portion, wherein the inner body is positioned within the cavity of the housing such that the housing and the inner body are movable relative to each other; and a biasing element positioned between the first end of the housing and the inner body; wherein, in a first position of the inner body, the first end of the inner body is longitudinally aligned with the first end of the housing such that biasing of at least a portion of the wall away from the longitudinal axis is resisted by the housing, and in a second position of the inner body, the second end of the housing is moved away from the second end portion of the inner body such that the first end of the inner body is longitudinally spaced apart from the first end of the housing and the at least a portion of the wall can be biased away from the longitudinal axis.

In some instances, the present disclosure provides a coupling device assembly comprising: a housing having a first end, a second end, and an inner surface forming a cavity, wherein the first end comprises a first opening into the cavity, and the second end comprises a second opening into the cavity; an inner body having a first end portion, a second end portion, a fluid passage extending through the first and second end portions of the inner body, the first end portion comprising a coupling sleeve formed by a wall having an inner surface that extends around a longitudinal axis defined by the fluid passage to form a recess extending from a first end of the inner body toward the second end portion, wherein the inner body is positioned within the cavity of the housing such that the housing and the inner body are movable relative to each other; a biasing element positioned between the first end of the housing and the inner body; and a tubing extending through the second opening of the housing and into the cavity, wherein an end portion of the tubing is coupled to the fluid passage of the inner body, wherein, in a first position of the inner body, the first end of the inner body is longitudinally aligned with the first end of the housing such that biasing of at least a portion of the wall away from the longitudinal axis is resisted by the housing, and in a second position of the inner body, the second end of the housing is moved away from the second end portion of the inner body such that the first end of the inner body is longitudinally spaced apart from the first end of the housing and the at least a portion of the wall can be biased away from the longitudinal axis.

In some instances, the present disclosure provides methods for providing a coupling device for medical tubing, the method comprising: inserting an inner body into a cavity of a housing such that the housing and the inner body are movable relative to each other, and a coupling sleeve defined by a proximal portion of the inner body extends through a first opening at a first end of the housing; inserting a biasing element into the housing such that the biasing element is positioned between a first end of the housing and the inner body; inserting a tubing through a second opening at a second end of the housing, and coupling the tubing to a fluid passage extending through the inner body; wherein, in a first position of the inner body, a first end of the inner body is longitudinally aligned with the first end of the housing such that biasing of at least a portion of coupling sleeve away from a longitudinal axis of the fluid passage of the inner body is resisted by the housing, and in a second position of the inner body, the second end of the housing is moved away from the second end of the inner body such that the first end of the inner body is longitudinally spaced apart from the first end of the housing and the at least a portion of the coupling sleeve can be biased away from the longitudinal axis.

Accordingly, the present application addresses several operational challenges encountered in prior fluid fitting connections and provides numerous improvements that enable the user to more easily and precisely connect and disconnect fluid fittings.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a coupling device for medical tubing in use with an IV set coupled to a patient, in accordance with aspects of the present disclosure.

FIG. 2 illustrates a perspective view of a coupling device for medical tubing, in a first configuration, in accordance with aspects of the present disclosure.

FIG. 3 illustrates a perspective view of the coupling device for medical tubing of FIG. 2, in a second configuration, in accordance with aspects of the present disclosure.

FIG. 4 illustrates an exploded perspective view of a coupling device for medical tubing in accordance with aspects of the present disclosure.

FIG. 5 is a cross-sectional view of a housing through the line 5-5 of FIG. 4, in accordance with aspects of the present disclosure.

FIG. 6 illustrates a partial cross-sectional perspective front view of a coupling device for medical tubing in accordance with aspects of the present disclosure.

FIG. 7 illustrates a partial cross-sectional perspective rear view of the coupling device for medical tubing in accordance with aspects of the present disclosure.

FIG. 8 illustrates cross-sectional side view of the coupling device for medical tubing of FIG. 2 in accordance with aspects of the present disclosure.

FIG. 9 illustrates a partial cross-sectional perspective front view of the coupling device for medical tubing of FIG. 3 in accordance with aspects of the present disclosure.

FIG. 10 illustrates cross-sectional side view of a coupling device for medical tubing, in a third configuration, in accordance with aspects of the present disclosure.

FIG. 11 illustrates cross-sectional side view of a coupling device for medical tubing, in a first configuration, in accordance with aspects of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present application discloses various features and advantages of a coupling device for medical tubing. The coupling device for medical tubing can provide for efficient and safe maintenance of fluid connections, such as the connections used for transferring medical fluids toward or away from a patient. The coupling device can maintain the fluid connection of a medical tubing by resisting disconnection when a pulling or tension force is applied to the coupling device, such as when a patient moves or when the medical tubing is pulled away from the patient. The coupling device also prevents injury to a patient or a caregiver by permitting disconnection of the fluid connection when a pulling or tension force exceeds a threshold. The coupling device also provides for efficient and safe reconnection of the fluid line by permitting the coupling device to be reconnected to the fluid transfer device, such as a catheter or needleless connector, thereby reestablishing the fluid pathway without requiring replacement of the coupling device.

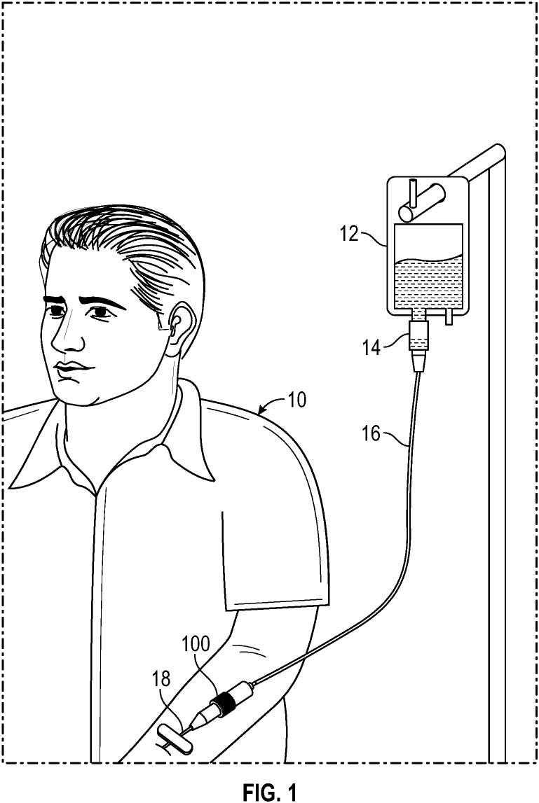

Referring now to the figures, FIG. 1 illustrates an example of a coupling device for medical tubing in use in accordance with aspects of the present disclosure. The coupling device 100 is coupled with tubing of an IV set, which is being used to direct a fluid to a patient 10. The IV set can include a medicament bag 12, a drip chamber 14, tubing 16, and an IV catheter 18.

The coupling device 100 fluidly connects the tubing 16 to the IV catheter 18. Although the coupling device 100 is illustrated being coupled along a fluid pathway of an IV set, between a medicament bag 12 and a patient 10, it should be understood that the coupling device 100 can be connected within other fluid pathways, such as between a patient and a IV pump or between a patient and a dialysis machine. The coupling device 100 can also be connected along another portion of a fluid pathway. For example, the coupling device 100 can be connected along a proximal portion of the fluid pathway, such as being connected between the tubing 16 and the medicament bag 12 or other fluid therapy device.

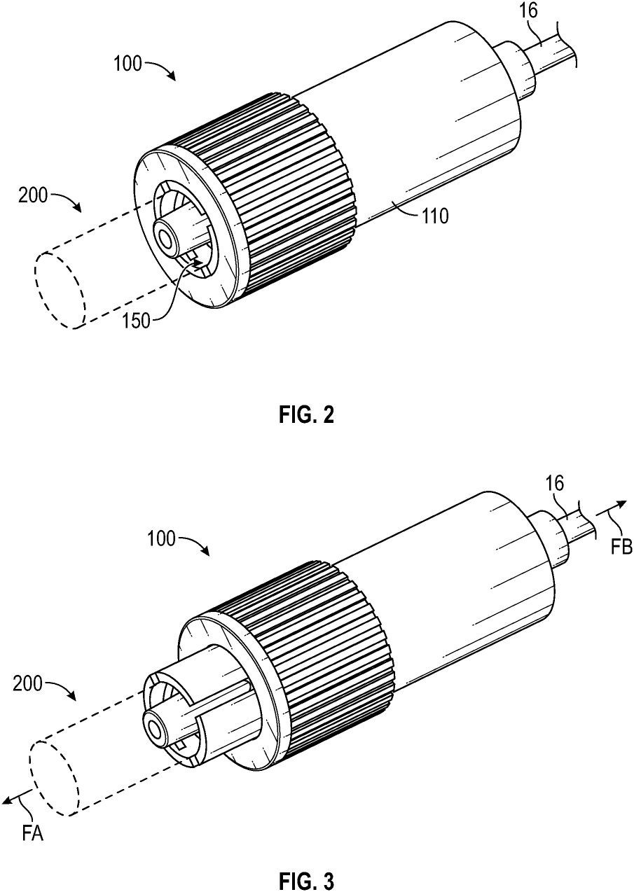

The coupling device 100 is configured to have a first configuration, a second configuration, and a third configuration. In the first configuration, which is illustrated in FIG. 2, the coupling device 100 can be coupled with a mating connector 200 to establish a fluid pathway between a tubing and the mating connector. For clarity and brevity, the mating connector 200, is illustrated in a general manner with broken lines.

The coupling device 100 includes a housing 110 and an inner body 150 positioned, at least partially, within an inner cavity of the housing 110. The inner body 150 includes a portion forming a coupling sleeve configured to couple with the mating connector 200. The coupling sleeve can include any structure configured to couple with a mating connector 200, such as a thread, a barb, or an interference fit between the inner body 150 and the mating connector 200.

When the coupling device 100 is in the first configuration, the inner body 150 is in a first position within the cavity of the housing 110, whereby the coupling sleeve of the inner body 150 resists separation or disconnection of the mating connector 200 from the coupling device 100.

In some embodiments of the present disclosure, when the inner body 150 is in the first position, an end of the inner body is approximately aligned or coplanar with an end of the housing. In some embodiments, a portion of the coupling sleeve of the inner body 150 or another portion of the inner body 150 may extend through the end of the housing by a first distance when the inner body 150 is in the first position.

When the coupling device 100 is in the first orientation, with the inner body 150 in the first position, the coupling device 100 can resist unintended separation of the mating connector 200 from the coupling device 100 by resisting biasing or flexing of the coupling sleeve away from mating connector 200. In some embodiments of the present disclosure, biasing of the coupling sleeve radially outward is resisted by the housing 110, such that, when the mating connector 200 and the coupling device 100 are pulled away from each other, the sleeve will engage against a portion of the housing 110, thereby resisting movement of the mating connector 200 out of the sleeve.

The coupling device 100 can be moved from the first configuration to the second configuration, which is illustrated in FIG. 3, when a pulling or tension force exceeds a threshold of the coupling device 100. For example, when a patient moves in an unintended manner, or when the medical tubing is pulled away from the patient, a pulling or tension force FA, FB may be applied to the mating connector 200 or the tubing 16 in a direction away from the coupling device 100.

If the force FA, FB exceeds a first threshold of the coupling device 100, any of the housing 110 and the inner body 150 can move away from each other so that the inner body 150 moves from the first position to a second position. The movement of the housing 110 and the inner body 150, relative to each other, can be along a longitudinal axis of the inner body 150 that extends between the mating connector 200 and an end portion of the tubing 16 connected to the coupling device 100.

In the second position of the inner body 150, at least a portion of the sleeve extends out of the cavity and away from an end of the housing 110 by a second distance. The second distance is greater than the first distance between the distal end of the sleeve and the end of the housing when the inner body 150 is in the first position.

If a pulling or tension force FA, FB exceeds a second threshold of the coupling device 100, the coupling sleeve of the inner body 150 is configured such that the engagement of the mating connector 200 against the sleeve causes at least a portion of the sleeve to bias or flex away from the mating connector 200. When the coupling sleeve flexes away from the mating connector 200, the resistance to separation of the coupling device 100 from the mating connector 200 is reduced, relative to when the coupling device 100 is in the first configuration, thereby permitting the coupling device 100 to separate from the mating connector 200. In some embodiments, engagement of a thread of the mating connector 200 against a thread of the inner body 150 when the force FA, FB exceeds the second threshold causes the portion of the sleeve to flex away from the mating connector 200. In some aspects of the present disclosure, a thread of the coupling device 100 can skip over or pass a thread of the mating connector 200 when the force FA, FB exceeds a second threshold of the coupling device 100.

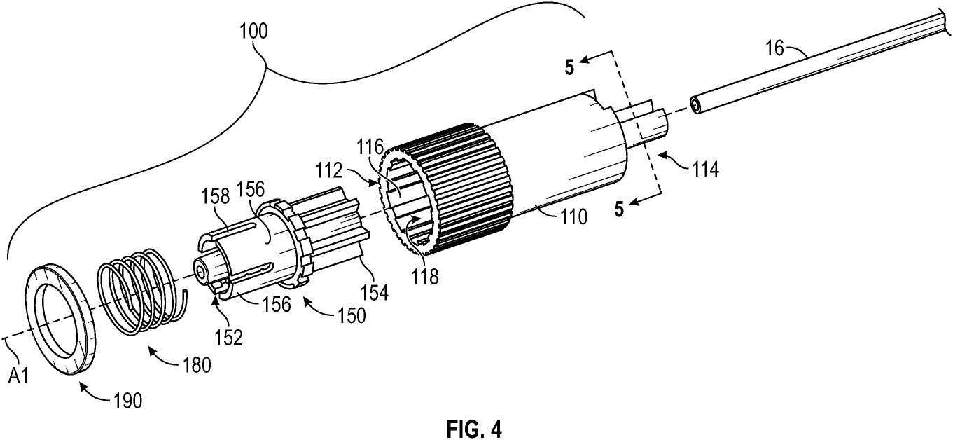

FIG. 4 illustrates an exploded perspective view of the coupling device 100. The coupling device 100 includes a housing 110, an inner body 150, a biasing element 180, and a cap 190. The inner body 150 and the biasing element 180 can be inserted into the housing 110, and a cap 190 can be coupled to the housing 110 to retain the inner body 150 within the housing 110, and the biasing element 180 between the inner body 150 and the cap 190.

Before inserting the inner body 150 into the housing 110, a length of tubing 16 can be inserted through an opening or tubing channel of the housing 110 and moved toward the inner body 150. An end of the tubing 16 can be coupled to the inner body 150, and a portion or length of the tubing 16 that intersects the opening of the housing 110 can be coupled to the housing. In some embodiments of the present disclosure, the end of the tubing is hermetically sealed to the inner body. Additionally, in some embodiments of the present disclosure, the portion of the tubing that intersects the opening of the housing 110, which is spaced apart from the end of the tubing, is coupled to the second end of the housing by an interference fit.

The housing 110 has a first end 112 and a second end 114, where the second end 114 is opposite to the first end 112. An inner surface 116 of the housing forms a cavity 118 that extends between the first end 112 and the second end 114 of the housing and is configured to receive the inner body 150 therein.

The first end 112 of the housing, or a portion of the housing adjacent to the first end 112, forms a first opening of the housing which is configured to permit the inner body 150 to be inserted through the first opening into the cavity. The second end 114 of the housing, or a portion of the housing adjacent to the second end 114, forms a second opening of the housing which is configured permit tubing to be positioned through the second opening into the cavity.

In some embodiments of the present disclosure, the inner surface 116 of the housing forms any of a ridge and/or groove that extending between the first and second ends 112, 114 of the housing. In some aspects of the present disclosure, a groove 130 is formed between adjacent ridges 132 and extends approximately parallel to a longitudinal axis A1 of the housing, where the longitudinal axis A1 is defined between the first and second ends 112, 114 of the housing.

The groove 130 and/or ridge 132 is configured to engage against the inner body 150 such that, when the housing 110 is rotated around the longitudinal axis A1, the groove 130 and/or ridge 132 can engage against the inner body 150, thereby rotating the inner body 150 with the housing 110. By rotationally coupling the housing 110 and the inner body 150, the housing 110 can be rotated to engage threads of the inner body 150 with complementary threads of a mating connector 200.

The inner body 150 has a first end portion 152, a second end portion 154, a fluid passage extending through the first and second end portions of the inner body. The first end portion 152 of the inner body forms a coupling sleeve bounded by a wall 156 of the inner body. The wall 156 has an inner surface that extends around a longitudinal axis defined by the fluid passage to form a recess extending from a first end of the inner body toward the second end portion.

At least one channel 158 extends through the wall 156 in a direction from a first end of the inner body toward the second end portion 154 of the inner body. In some embodiments of the present disclosure, two or more channels 158 extend through the wall 156 of the inner body to form a plurality of flexible fingers, wherein each finger is defined between adjacent channels 158. As such, the inner body 150 can include a first flexible finger and a second flexible finger. In some aspects of the present disclosure, the inner body 150 includes four flexible fingers, which are separated from each other by four channels through the wall 156.

The first end portion 152 of the inner body, along the one or more channel 158, is more flexible, relative to another portion of the inner body that is spaced apart from the first end portion 152. In some embodiments, the first end portion 152 of the inner body is more flexible than the second end portion 154 of the inner body.

A biasing element 180 is positioned in the cavity 118 of the housing to direct the inner body 150 in the cavity 118 toward the second end 114 of the housing. The biasing element 180 can be any of a spring, a resilient bellows, or any similar structure configured to direct the inner body 150 toward the second end 114 of the housing. In some embodiments of the present disclosure, the biasing element 180 can be positioned between the inner body 150 and the second end 114 of the housing, so that the biasing element can resist movement of the inner body 150 toward the first end 112 of the housing.

A cap 190 is coupled to the first end 112 of the housing to retain the biasing element 180 and the inner body 150 within the cavity 118 of the housing. The cap 190 includes a passage defining at least a portion of the first opening through the first end 112 of the housing. The passage through the cap 190 can have a cross-sectional width that is less than a cross-sectional width formed by the cavity 118 of the housing such that the cap 190 can resist movement of the biasing element 180 out of the cavity 118 and permit the inner body to move out of the cavity 118 through the passage of the cap 190.

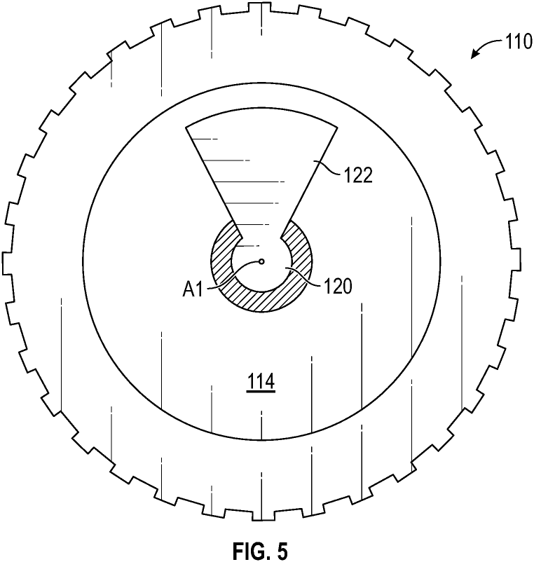

FIG. 5 illustrates a rear cross-sectional view of the housing 110 in the direction of 5-5 in FIG. 4. As illustrated, the housing 110 includes a second opening 120 through the second end 114 and into the cavity of the housing. The second opening 120 is formed by an inner surface of the housing that extends around the longitudinal axis A1 of the housing. In some embodiments of the present disclosure, the inner surface of the housing that forms the second opening 120 extends around approximately three-quarters of the longitudinal axis A1.

A cross-sectional width of the second opening 120 can be approximately equal to or less than a cross-sectional width or diameter defined by the outer surface of the tubing. The cross-sectional width of the second opening 120 is configured to permit the tubing 16 to be inserted into the second opening 120 while forming an interference fit that can resist any of a longitudinal and/or a torsional movement of the tubing relative to the inner surface of the housing that forms the second opening 120.

The housing 110 can also include a tubing channel 122 configured to permit insertion of the tubing through the tubing channel 122 and into the cavity with minimal or no resistance. The tubing channel 122 extends through the second end 114 and into the cavity of the housing and intersects the second opening 120.

In some embodiments of the present disclosure, the tubing channel 122 comprises a cross-sectional width that tapers or decreases toward the second opening 120. To permit the tubing to be inserted through the tubing channel 122, the tubing channel 122 can have a maximum cross-sectional width that is equal to or greater than a cross-sectional width or diameter defined by the outer surface of the tubing.

In use, an end portion of the tubing can be inserted through the tubing channel 122 and into the cavity 118 and moved through the first end 112 of the housing. The end of the tubing can then be coupled to the fluid passage of the inner body 150. Before or after inserting the inner body 150 into the cavity 188 of the housing, the portion or length of the tubing that intersects the second end 114 of the housing can be moved into the second opening 120 to couple the tubing to the housing 110.

In some embodiments of the present disclosure, the end of the tubing is hermetically sealed to the inner body. Additionally, in some embodiments of the present disclosure, a portion or length of the tubing is coupled to the housing at the second opening 120 by an interference fit. The inner surface of the second opening, in some embodiments, can engage against the outer surface of the tubing that extends around approximately three-quarters of the circumference of the tubing.

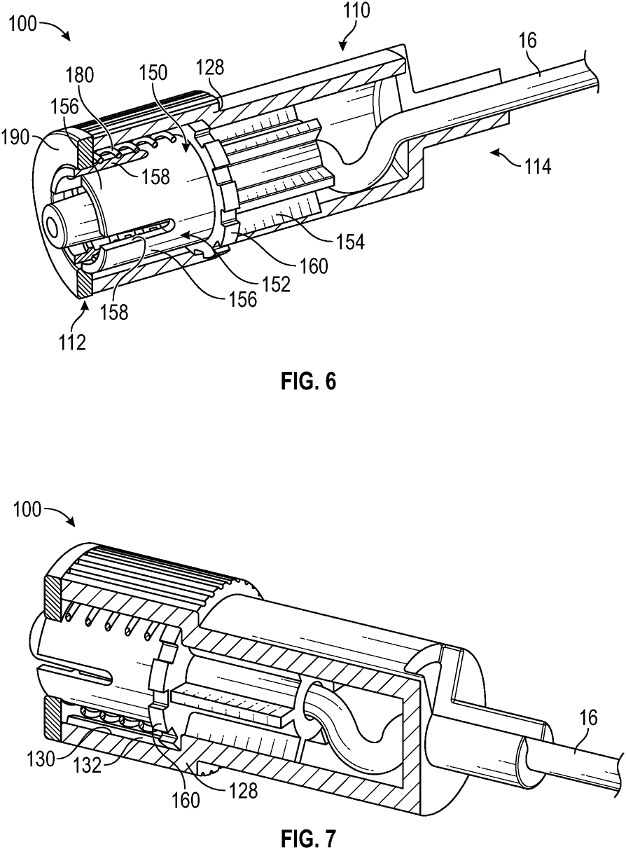

FIGS. 6 through 8 illustrate the coupling device 100 in the first configuration with the inner body 150 in the first position. In the first position, the inner body 150 is located within the cavity 118 of the housing such that the first end portion 152 of the inner body is adjacent to the first end 112 of the housing, and the second end portion 154 of the inner body is adjacent to the second end 114 of the housing.

The tubing 16 is coupled to the inner body 150 and to the housing 110 so that a length L1 of the tubing, between the inner body 150 and the distal end of cavity 118, is greater than a distance D1 between the inner body 150 and the distal end of cavity 118. Because the length L1 of the tubing 16 within the cavity 118 is longer than the distance D1 between the inner body 150 and the distal end of cavity 118, movement of the inner body 150 away from the second end 114 of the housing may not be resisted by the tubing 16. The portion of the tubing 16 within the cavity 118 also permits movement of the inner body 150 away from the second end 114 without altering the volume of the fluid pathway through the coupling device 100, thereby providing a neutral displacement coupling device 100.

The inner body 150 is configured to be moveable within the cavity 118, between the first and second ends 112, 114 of the housing. When the inner body 150 is in the first position, as illustrated in FIGS. 6 through 8, a first end of the inner body 150 is longitudinally aligned with the first end 112 of the housing. If the wall 156 of the inner body is biased radially outward, away from the longitudinal axis A1, the wall 156 will engage against the housing or cap 190, thereby resisting biasing of the wall 156 and resisting separation of a mating connector from the inner body 150.

The inner body 150 is biased or directed toward the second end 114 of the housing by the biasing element 180. The biasing element 180 can be a spring positioned between the first end 112 of the housing and the second end portion 154 of the inner body. The biasing element 180 can be configured to extend around the outer surface of the inner body 150.

In some embodiments, the inner body 150 comprises a spline 160 that extends from an outer surface of the inner body, in a direction that is radially outward away from an outer surface of the inner body.

The housing includes a ledge 128 that is configured to resist movement of the inner body 150 toward the second end 114 of the housing. The ledge 128 is formed by a portion of the cavity 118, adjacent to the second end 114 of the housing, having a smaller cross-sectional width relative to a portion of the cavity 118 adjacent to the first end 112 of the housing. In some embodiments, the ledge 128 can be formed by the cross-sectional width of the cavity 118 tapering or decreasing in a direction toward the second end 114 of the housing. In some embodiments of the present disclosure, the ledge 128 is formed by a portion of the housing that extends radially inward from the inner surface 116 into the cavity 118 of the housing.

In the first configuration of the coupling device 100 with the inner body 150 in the first position, the biasing element 180 is positioned between the cap 190 and the spline 160 such that the inner body 150 is directed toward the second end 114 of the housing. Movement of the inner body 150 toward the second end 114 of the housing is limited by engagement of the spline 160 against the ledge 128.

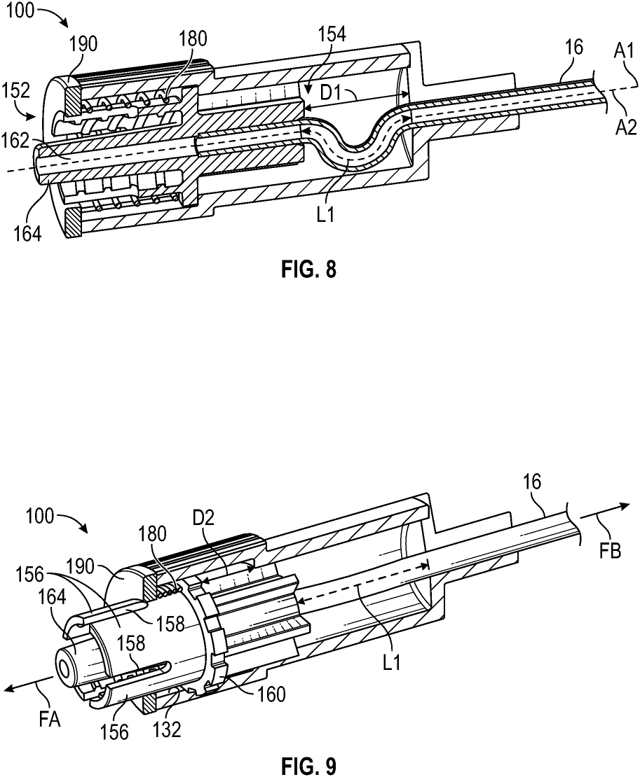

The fluid passage 162 through the inner body, is shown, for example, in at least FIG. 8. The fluid passage 162 extends through the first and second end portions of the inner body. A portion of the fluid passage 162, along the first end portion 152 of the inner body, is configured to fluidly couple with a fluid pathway of mating connector, while another portion of the fluid passage 162, along the second end portion 154 of the inner body, is configured to fluidly couple with a length of tubing 16. The fluid passage 162 defines a longitudinal axis A2 that extends through the first and second end portions of the inner body.

The portion of the fluid passage 162 along the first end portion 152 of the inner body can be formed, at least in part, by a protrusion of the inner body. In some embodiments of the present disclosure, the protrusion of the inner body forming a portion of the fluid passage that extends within or through the recess defines a luer tip 164. In some aspects, the luer tip comprises an outer surface having a cross-sectional width that tapers or decreases in a direction away from the second end portion 154 of the inner body. In some embodiments of the present Application, the luer tip 164 can include a valve configured to resist a fluid flow through the fluid passage 162 when the coupling device 100 is not coupled with a mating connector.

The inner body 150 can include a thread that extends along the inner surface of the wall 156, including along the flexible fingers formed between the channels 158. The thread of the inner body 150 is configured to engage against a complementary thread of a mating connector. When the coupling device 100 is in the first and second orientations, the thread can resist separation of the coupling device 100 from the mating connector.

In some embodiments of the present disclosure, the coupling device 100 can have other structures or features for coupling with a mating connector, such as a key and/or slot configured to couple with a complementary key and/or slot of a mating connector. It is also contemplated that the coupling device 100 can couple with a mating connector by means of an interference fit of the inner surface of the sleeve of the coupling device engaged against an outer surface of the mating connector.

FIG. 9 illustrates the coupling device 100 is in the second orientation, with the inner body 150 in the second position. The coupling device 100 can be moved to the second orientation when any of the housing 110 and/or the inner body 150 are moved in a direction away from each other. The coupling device 100 can be moved to the second orientation when the inner body 150 is pulled away from the housing 110, such as when the mating connector is pulled away from the coupling device 100, thereby creating a force on the inner body 150 in a direction away from the housing 110, as shown by arrow FA. The coupling device 100 can be moved to the second orientation when the tubing 16 is pulled away from the coupling device 100, thereby creating a force on the housing 110 in a direction away from the inner body 150, as shown by arrow FB.

When the force on the housing 110 and/or the inner body 150 exceeds a first threshold, the coupling device 100 is configured so that the housing 110 and the inner body 150 can move away from each other, and the biasing element 180 is compressed between the first end of the housing 112 and the first end portion 152 of the inner body.

In some embodiments, each of the housing 110 and the inner body 150 move, relative to each other, along their respective longitudinal axis A1, A2. In some embodiments, the longitudinal axis A1 of the housing 110 and the longitudinal axis A2 of the inner body 150 are aligned to form a common axis.

As the housing 110 and the inner body 150 move, relative to each other, each of the housing 110 and the inner body 150 remain rotationally coupled by the spline 160 of the inner body positioned within the groove 130 of the housing.

The housing 110 and the inner body 150 are configured to move away from each other by a distance D2. The distance D2 can be less than the length L1 of the tubing 16 between the inner body 150 and the distal end of cavity 118 so that the volume of the fluid pathway through the coupling device 100 may remain constant when the coupling device moves between the first and second configurations.

When the coupling device 100 is in the second orientation, the coupling sleeve formed by the first end portion 152 of the inner body extends out of the cavity 118, in a direction away from the first end 112 of the housing. As such, the wall 156, or a portion thereof, is not aligned with the first end 112 of the housing. Thus, the wall 156 can move or be flexed radially outward in a direction away from the longitudinal axis A2 without engaging against the housing 110.

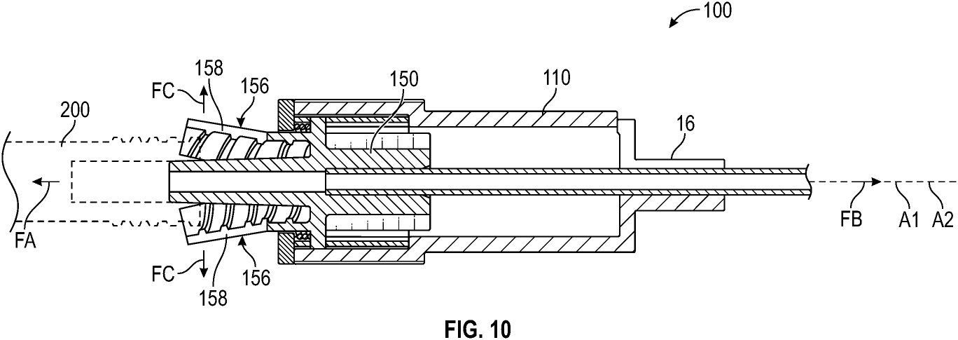

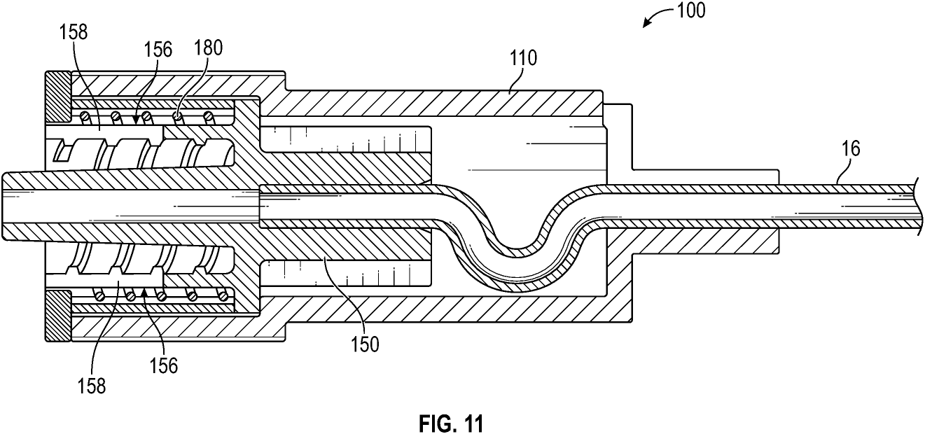

If a pulling or tension force FA, FB exceeds a second threshold of the coupling device 100, the coupling device 100 can move from the second orientation to a third orientation, as illustrated in FIG. 10. In the third orientation, the resistance to separation of the mating connector 200 from the coupling device 100 is reduced, relative to when the coupling device 100 is in the first or second configuration. In the third orientation, the mating connector 200 can move away from the inner body 150, thereby permitting separation of the mating connector 200 from the coupling device 100.

The coupling device 100 can move from the second configuration to the third configuration when a pulling or tension force FA, FB between the mating connector 200 and the coupling device 100 exceeds a second threshold. The force FA, FB can cause a force FC to be exerted from the mating connector 200 against the wall 156 in a direction away from the longitudinal axis A2, which can cause the wall 156 or another portion of the coupling sleeve to be moved or flexed away from the longitudinal axis A2.

In the third orientation, a cross-sectional width defined by the inner surface of the coupling sleeve is approximately equal to or greater than a cross-sectional width defined by the outer surface of the mating connector 200. In some embodiments, when the coupling device 100 is in the third orientation, the thread of the coupling device 100 can skip over or pass a thread of the mating connector 200, thereby reducing the resistance to separation of the coupling device 100 from the mating connector 200.

After the pulling or tension force FA, FB is reduced to less than the first threshold, the inner body 150 can move from the second position to the first position so that the coupling device 100 is in the first configuration. The inner body 150 can move from the second position to the first position so that the coupling device 100 is in the first configuration when the pulling the tension force FA, FB is reduced or when the mating connector 200 is separated from the coupling device 100 such that the pulling the tension force FA, FB is no longer present. When the pulling the tension force FA, FB is reduced to less than the first threshold, the biasing element 180 directs the inner body 150 toward the second end 114 of the housing.

In some embodiments of the present disclosure, the first threshold is greater than a force exerted by the biasing element 180 against the inner body 150 in the direction toward the second end 114 of the housing. In some embodiments of the present disclosure, the first threshold is between approximately 2 pounds (0.91 kgs) and approximately 3 pounds (1.36 kgs). In some embodiments of the present disclosure, the first threshold is approximately 2.5 pounds (1.13 kgs). The second threshold can be greater than the first threshold. In some embodiments of the present disclosure, the second threshold is between approximately 3 pounds (1.36 kgs) and approximately 7 pounds (3.17 kgs). In some embodiments of the present disclosure, the first threshold is approximately 5 pounds (2.27 kgs).

When the coupling device 100 is in the first configuration, such as when the coupling device 100 returns to the first configuration after a mating connector 200 is disconnected from the coupling device 100, the coupling device 100 can be reconnected to the same or another mating connector.

Accordingly, the coupling device 100 can provide for efficient and safe maintenance of fluid connections, can maintain the fluid connection of a medical tubing, and can permit disconnection and reconnection of the fluid pathway with a patient.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A coupling device for medical tubing, the coupling device comprising: a housing having an inner surface forming a cavity, a first opening into the cavity, and a second opening into the cavity; and an inner body having a fluid passage, extending through the inner body, and a coupling sleeve, wherein the inner body is positioned within the cavity of the housing with the coupling sleeve extending toward the first opening of the housing such that the housing and the inner body are movable relative to each other; wherein, in a first position of the inner body, biasing of at least a portion of the coupling sleeve in a direction that is radially outward is resisted by engagement of the coupling sleeve with the housing; and wherein, in a second position of the inner body, the coupling sleeve is moved through the first opening in a direction that is away from the second opening of the housing such that the at least a portion of the coupling sleeve can be biased radially outward.

Clause 2. The coupling device of Clause 1, wherein the inner body comprises at least one channel that extends through the coupling sleeve in a direction from a first end of the inner body toward a second end of the inner body.

Clause 3. The coupling device of Clause 2, wherein the at least one channel comprises a first channel and a second channel, wherein the second channel is spaced apart from the first channel, and where a first portion of the coupling sleeve between the first and second channels forms a first flexible finger, and a second portion of the coupling sleeve between the first and second channels forms a second flexible finger.

Clause 4. The coupling device of Clause 1, wherein the inner body comprises a luer tip that extends within the coupling sleeve, toward a first end of the inner body.

Clause 5. The coupling device of Clause 1, further comprising a biasing element positioned between a first end of the housing and the inner body.

Clause 6. The coupling device of Clause 1, wherein the inner body comprises a spline that extends from an outer surface of the inner body, in a direction that is radially outward away from an outer surface of the inner body.

Clause 7. The coupling device of Clause 6, wherein the spline is configured to be positioned within a groove extending along the inner surface of the housing such that the housing and the inner body are rotationally coupled.

Clause 8. The coupling device of Clause 7, wherein the housing and the inner body are movable, relative to each other, along a length of the groove.

Clause 9. A coupling device for medical tubing, the coupling device comprising: a housing having a first end, a second end, and an inner surface forming a cavity, wherein the first end comprises a first opening into the cavity, and the second end comprises a second opening into the cavity; an inner body having a first end portion, a second end portion, a fluid passage extending through the first and second end portions of the inner body, the first end portion comprising a coupling sleeve formed by a wall having an inner surface that extends around a longitudinal axis defined by the fluid passage to form a recess extending from a first end of the inner body toward the second end portion, wherein the inner body is positioned within the cavity of the housing such that the housing and the inner body are movable relative to each other; and a biasing element positioned between the first end of the housing and the inner body; wherein, in a first position of the inner body, the first end of the inner body is longitudinally aligned with the first end of the housing such that biasing of at least a portion of the wall away from the longitudinal axis is resisted by the housing, and in a second position of the inner body, the second end of the housing is moved away from the second end portion of the inner body such that the first end of the inner body is longitudinally spaced apart from the first end of the housing and the at least a portion of the wall can be biased away from the longitudinal axis.

Clause 10. The coupling device of Clause 9, wherein the inner body comprises at least one channel that extends through the wall, from the first end of the inner body toward the second end portion of the inner body.

Clause 11. The coupling device of Clause 10, wherein the at least one channel comprises a first channel and a second channel, wherein the second channel is spaced apart from the first channel, and where a first portion of the wall between the first and second channels forms a first flexible finger, and a second portion of the wall between the first and second channels forms a second flexible finger.

Clause 12. The coupling device of Clause 9, wherein the inner body comprises a thread that extends along the inner surface of the wall.

Clause 13. The coupling device of Clause 9, wherein the inner body comprises a luer tip that extends within the coupling sleeve, from a bottom surface of the recess toward the first end of the inner body.

Clause 14. The coupling device of Clause 9, wherein the first opening of the housing comprises a first cross-sectional width, and an outer surface of the wall of the inner body comprises a second cross-sectional width, and wherein the first cross-sectional width is equal to or greater than the second cross-sectional width, such that, the first end portion of the inner body is longitudinally movable between the first and second positions through the first opening.

Clause 15. The coupling device of Clause 9, wherein the inner body comprises a spline that extends from an outer surface of the inner body, in a direction that is radially outward away from the longitudinal axis.

Clause 16. The coupling device of Clause 15, wherein the spline is configured to be positioned within a groove extending along the inner surface of the housing such that the housing and the inner body are rotationally coupled.

Clause 17. The coupling device of Clause 16, wherein the housing and the inner body are movable, relative to each other, along a length of the groove.

Clause 18. The coupling device of Clause 9, wherein the housing comprise a ledge that extends radially inward from the inner surface into the cavity.

Clause 19. The coupling device of Clause 18, wherein, in the first position, a portion of the inner body is engaged against the ledge, thereby resisting movement of inner body toward the second end of the housing.

Clause 20. The coupling device of Clause 9, wherein the housing comprises a tubing channel that extends through a second end of the housing and intersects the second opening..

Clause 21. The coupling device of Clause 20, wherein at least a portion of the tubing channel comprises a cross-sectional width that is greater than a cross-sectional width of the second opening.

Clause 22. The coupling device of Clause 21, wherein the cross-sectional width of the tubing channel decreases in a direction toward the second opening.

Clause 23. The coupling device of Clause 9, wherein a distance from the first end of the inner body to a second end of the inner body is greater than a distance from the first end of the housing to a distal end of the cavity.

Clause 24. The coupling device of Clause 9, further comprising a cap coupled to a first end of the housing, the cap forming at least a portion of the first opening of the housing.

Clause 25. The coupling device of Clause 9, wherein the biasing element comprises a spring configured to direct the inner body toward the first position.

Clause 26. A coupling device assembly comprising: a housing having a first end, a second end, and an inner surface forming a cavity, wherein the first end comprises a first opening into the cavity, and the second end comprises a second opening into the cavity; an inner body having a first end portion, a second end portion, a fluid passage extending through the first and second end portions of the inner body, the first end portion comprising a coupling sleeve formed by a wall having an inner surface that extends around a longitudinal axis defined by the fluid passage to form a recess extending from a first end of the inner body toward the second end portion, wherein the inner body is positioned within the cavity of the housing such that the housing and the inner body are movable relative to each other; a biasing element positioned between the first end of the housing and the inner body; and a tubing extending through the second opening of the housing and into the cavity, wherein an end portion of the tubing is coupled to the fluid passage of the inner body, wherein, in a first position of the inner body, the first end of the inner body is longitudinally aligned with the first end of the housing such that biasing of at least a portion of the wall away from the longitudinal axis is resisted by the housing, and in a second position of the inner body, the second end of the housing is moved away from the second end portion of the inner body such that the first end of the inner body is longitudinally spaced apart from the first end of the housing and the at least a portion of the wall can be biased away from the longitudinal axis.

Clause 27. The coupling device assembly of Clause 26, wherein, when the inner body is in the first position, a length of the tubing between a second end of the inner body and a distal end of the cavity, adjacent to the second end of the housing, is longer than a distance between the second end of the inner body and the distal end of the cavity.

Clause 28. A method for providing a coupling device for medical tubing, the method comprising: inserting an inner body into a cavity of a housing such that the housing and the inner body are movable relative to each other, and a coupling sleeve defined by a proximal portion of the inner body extends through a first opening at a first end of the housing; inserting a biasing element into the housing such that the biasing element is positioned between a first end of the housing and the inner body; inserting a tubing through a second opening at a second end of the housing, and coupling the tubing to a fluid passage extending through the inner body; wherein, in a first position of the inner body, a first end of the inner body is longitudinally aligned with the first end of the housing such that biasing of at least a portion of coupling sleeve away from a longitudinal axis of the fluid passage of the inner body is resisted by the housing, and in a second position of the inner body, the second end of the housing is moved away from the second end of the inner body such that the first end of the inner body is longitudinally spaced apart from the first end of the housing and the at least a portion of the coupling sleeve can be biased away from the longitudinal axis.

Clause 29. The method of Clause 28 wherein, when the inner body is in the second position and a mating connector, coupled to the coupling sleeve, is moved in a direction away from the first end of the inner body, engagement of the mating connector with the coupling sleeve directs at least a portion of the coupling sleeve away from the longitudinal axis.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. §101, 102, or 103, nor should they be interpreted in such a way.

What is claimed is:

1. A coupling device for medical tubing, the coupling device comprising:
a housing having an inner surface forming a cavity, a first opening into the cavity, and a second opening into the cavity; and
an inner body having a fluid passage, extending through the inner body, and a coupling sleeve, wherein the inner body is positioned within the cavity of the housing with the coupling sleeve extending toward the first opening of the housing such that the housing and the inner body are movable relative to each other;
wherein, in a first position of the inner body, biasing of at least a portion of the coupling sleeve in a direction that is radially outward is resisted by engagement of the coupling sleeve with the housing; and wherein, in a second position of the inner body, the coupling sleeve is moved through the first opening in a direction that is away from the second opening of the housing such that the at least a portion of the coupling sleeve can be biased radially outward.

2. The coupling device of claim 1, wherein the inner body comprises at least one channel that extends through the coupling sleeve in a direction from a first end of the inner body toward a second end of the inner body.

3. The coupling device of claim 2, wherein the at least one channel comprises a first channel and a second channel, wherein the second channel is spaced apart from the first channel, and where a first portion of the coupling sleeve between the first and second channels forms a first flexible finger, and a second portion of the coupling sleeve between the first and second channels forms a second flexible finger.

4. The coupling device of claim 1, wherein the inner body comprises a luer tip that extends within the coupling sleeve, toward a first end of the inner body.

5. The coupling device of claim 1, further comprising a biasing element positioned between a first end of the housing and the inner body.

6. The coupling device of claim 5, wherein the biasing element comprises a spring configured to direct the inner body toward the first position.

7. The coupling device of claim 1, wherein the inner body comprises a spline that extends from an outer surface of the inner body, in a direction that is radially outward away from an outer surface of the inner body.

8. The coupling device of claim 7, wherein the spline is configured to be positioned within a groove extending along the inner surface of the housing such that the housing and the inner body are rotationally coupled.

9. The coupling device of claim 8, wherein the housing and the inner body are movable, relative to each other, along a length of the groove.

10. The coupling device of claim 1, wherein the inner body comprises a thread that extends along the inner surface of the coupling sleeve.

11. The coupling device of claim 1, wherein the first opening of the housing comprises a first cross-sectional width, and an outer surface of the coupling sleeve comprises a second cross-sectional width, and wherein the first cross-sectional width is equal to or greater than the second cross-sectional width, such that, a first end of the inner body is longitudinally movable between the first and second positions through the first opening.

12. The coupling device of claim 1, wherein the housing comprise a ledge that extends radially inward from the inner surface into the cavity.

13. The coupling device of claim 1, wherein the housing comprises a tubing channel that extends through a second end of the housing and intersects the second opening.

14. The coupling device of claim 13, wherein at least a portion of the tubing channel comprises a cross-sectional width that is greater than a cross-sectional width of the second opening.

15. The coupling device of claim 1, wherein a distance from a first end of the inner body to a second end of the inner body is greater than a distance from the first end of the housing to a distal end of the cavity.

16. The coupling device of claim 1, further comprising a cap coupled to a first end of the housing, the cap forming at least a portion of the first opening of the housing.

17. A coupling device assembly comprising:
a housing having a first end, a second end, and an inner surface forming a cavity, wherein the first end comprises a first opening into the cavity, and the second end comprises a second opening into the cavity;
an inner body having a first end portion, a second end portion, a fluid passage extending through the first and second end portions of the inner body, the first end portion comprising a coupling sleeve formed by a wall having an inner surface that extends around a longitudinal axis defined by the fluid passage to form a recess extending from a first end of the inner body toward the second end portion, wherein the inner body is positioned within the cavity of the housing such that the housing and the inner body are movable relative to each other;
a biasing element positioned between the first end of the housing and the inner body;and
a tubing extending through the second opening of the housing and into the cavity, wherein an end portion of the tubing is coupled to the fluid passage of the inner body,
wherein, in a first position of the inner body, the first end of the inner body is longitudinally aligned with the first end of the housing such that biasing of at least a portion of the wall away from the longitudinal axis is resisted by the housing, and in a second position of the inner body, the second end of the housing is moved away from the second end portion of the inner body such that the first end of the inner body is longitudinally spaced apart from the first end of the housing and the at least a portion of the wall can be biased away from the longitudinal axis.

18. The coupling device assembly of claim 17, wherein, when the inner body is in the first position, a length of the tubing between a second end of the inner body and a distal end of the cavity, adjacent to the second end of the housing, is longer than a distance between the second end of the inner body and the distal end of the cavity.

19. A method for providing a coupling device for medical tubing, the method comprising:
inserting an inner body into a cavity of a housing such that the housing and the inner body are movable relative to each other, and a coupling sleeve defined by a proximal portion of the inner body extends through a first opening at a first end of the housing;
inserting a biasing element into the housing such that the biasing element is positioned between a first end of the housing and the inner body;
inserting a tubing through a second opening at a second end of the housing, and coupling the tubing to a fluid passage extending through the inner body;
wherein, in a first position of the inner body, a first end of the inner body is longitudinally aligned with the first end of the housing such that biasing of at least a portion of coupling sleeve away from a longitudinal axis of the fluid passage of the inner body is resisted by the housing, and in a second position of the inner body, the second end of the housing is moved away from the second end of the inner body such that the first end of the inner body is longitudinally spaced apart from the first end of the housing and the at least a portion of the coupling sleeve can be biased away from the longitudinal axis.

20. The method of claim 19 wherein, when the inner body is in the second position and a mating connector, coupled to the coupling sleeve, is moved in a direction away from the first end of the inner body, engagement of the mating connector with the coupling sleeve directs at least a portion of the coupling sleeve away from the longitudinal axis.
